(19) United States
(12) Patent Application Publication
(10) Pub. No.: US 2026/0069772 A1
(43) Pub. Date: Mar. 12, 2026

(54) NEEDLE AND A METHOD OF TREATING A SURFACE OF A NEEDLE

(71) Applicant: Unomedical A/S, Lejre (DK)

(72) Inventor: Erik Othel-Jacobsen, Snekkersten (DK)

(21) Appl. No.: 19/393,878

(22) Filed: Nov. 19, 2025

Related U.S. Application Data
(63) Continuation of application No. PCT/EP2024/063812, filed on May 17, 2024.
(60) Provisional application No. 63/503,340, filed on May 19, 2023.
(60) Provisional application No. 63/503,350, filed on May 19, 2023.
(60) Provisional application No. 63/503,360, filed on May 19, 2023.

(30) Foreign Application Priority Data
Jul. 6, 2023 (GB) 2310425.0
Jul. 6, 2023 (GB) 2310429.2
Jul. 6, 2023 (GB) 2310432.6

(51) Int. Cl. A61M 5/158; A61L 29/02; B21G 1/00

(52) U.S. Cl. CPC A61M 5/158; A61L 29/02; B21G 1/006; A61M 2205/0266; A61M 2207/00

(57) ABSTRACT

The invention relates to a needle having a lumen for distributing fluid into, or removing fluid from, a soft tissue of a patient. The needle includes a nickel-titanium alloy. The needle is treated with an acidic solution including hydrofluoric acid at a temperature less than 60° C. for at least 10 seconds.

### TECHNICAL FIELD

The present invention relates to a needle, particularly a needle for distributing fluid into, or removing fluid from, a soft tissue, for example subcutaneous tissue, of a patient. The present invention also relates to a method of treating a surface of a nickel-titanium needle.

### BACKGROUND

Many current infusion devices, for example for continuous subcutaneous insulin infusion (CSII) therapy, utilise a soft cannula. Rigid insertion needles are required to enable the insertion of such cannulas into the patient's tissue. Insertion needles can add to the pain and tissue trauma associated with the use of such devices due to size differences between the needle and the cannula. In particular, the wound created by the needle is too small for the soft cannula. The cannula therefore typically needs to be pressed through the smaller hole, which will cause more pain to the patient. Moreover, many soft cannulas do not sufficiently resist kinking, which can stop the flow of the therapeutic agent. On the other hand, if the infusions are performed with a conventional needle, the tip of the rigid needle will engage the adjacent tissue during patient movement, which may worsen the wound and cause additional trauma to the patient. For these reasons among others, there remains a need for further improvements in this technological field.

Needles are available in a wide variety of configurations, and commonly include at least one bevel. A bevel is an angled surface formed at the tip of the needle when it is sharpened to make a needle point. Some needles include multiple bevels, for example a lancet needle—the most common needle used in medical applications—has three bevelled surfaces: the primary bevel, which is typically formed by grinding the tip at an angle, and two secondary bevels, which are typically formed by grinding opposite sides of the primary bevel to produce the needle point and a cutting edge. Diametrically opposite the needle point is the heel of the needle, which is sometimes referred to as the heel of the bevel.

During insertion, an arcuate cut is made in the tissue, extending from each side of the needle point as the needle advances. As the cut continues to extend, a hinged flap of tissue overlaying the lumen of the needle is formed. The heel may provide a cutting edge that further extends the cut such that the flap is severed, and subsequently the flap may be pushed into the lumen by further advancement of the needle. This undesirable mechanism is known as coring. Coring may similarly occur when a needle is inserted through a pharmaceutical closure, i.e. a bung or plug, of a container of medicament to draw up medicament therefrom, wherein a fragment of the material forming the closure is cut away. Coring is undesirable as it reduces the efficacy of the needle, and consequently the efficacy of delivery of medicament to a patient.

Coring may lead to contamination of medicament and/or injection of a foreign body, for example a severed fragment of the material forming pharmaceutical closure, into a patient. Coring may increase foreign body response (FBR) to the medical procedure in question. Coring has led to the development of non-coring needles, wherein the heel is sandblasted and thereby dulled to ensure the heel provides a non-cutting edge. This is an additional process step, and may be difficult or undesirable when processing certain materials used to manufacture needles. In particular, sandblasting may be difficult when manufacturing needles formed of a superelastic alloy, for example a nickel titanium alloy, i.e. nitinol, due to its high flexibility.

It would be useful to provide an improved needle for insertion into the soft tissue, for example the subcutaneous tissue, of a patent. In particular, it would be useful to provide a needle that reduces or minimises pain and tissue trauma and minimises FBR.

It would also be useful to provide a needle that is insertable into soft tissue and that ensures a reliable flow of fluids through the lumen of the needle.

It would be useful to provide a method for treating a needle in order to provide an improved needle for insertion into the soft tissue, for example the subcutaneous tissue, of a patent. In particular, it would be useful to provide a needle that reduces or minimises pain and tissue trauma and minimises FBR.

It would also be useful to provide a method for treating a needle in order to provide a needle that is insertable into soft tissue and that ensures a reliable flow of fluids through the lumen of the needle.

### BRIEF SUMMARY

The invention is set out in the appended claims.

According to an aspect of the invention there is a provided a needle having a lumen for distributing fluid into, or removing fluid from, a soft tissue, for example a subcutaneous tissue, of a patient.

The needle may comprise a nickel-titanium alloy. The nickel-titanium alloy may be superelastic or pseudoelastic.

The needle may be treated with an acidic solution, for example an acidic solution including hydrofluoric acid. Additionally or alternatively, the needle may be treated at a temperature less than 60° C. Additionally or alternatively, the needle may be treated for at least 10 seconds.

According to another aspect of the invention, there is provided a needle having a lumen for distributing fluid into, or removing fluid from, a soft tissue of a patient, the needle comprising a nickel-titanium alloy; wherein the needle is treated with an acidic solution including hydrofluoric acid at a temperature less than 60° C. for at least 10 seconds.

According to an aspect of the invention, there is provided a needle having a lumen for distributing fluid into, or removing fluid from, a soft tissue of a patient, the needle comprising a nickel-titanium alloy; wherein the needle is treated with an acidic solution including hydrofluoric acid at a temperature of at least 15° C. and less than 60° C. for at least 10 seconds.

In examples, the acidic solution includes at least 5 volume percent hydrofluoric acid. The acidic solution may include less than 50 volume percent hydrofluoric acid. The acidic solution may include, for example between 10 and 45 volume percent hydrofluoric acid.

In examples, the acidic solution includes nitric acid. The acidic solution may include at least 15 volume percent nitric acid. The acidic solution may include less than 70 volume percent nitric acid. The acidic solution may include, for example between 20 and 65 volume percent nitric acid.

In examples, the needle may be treated, for example with the acidic solution, at a temperature of at least 15° C. The needle may be treated at a temperature less than 55° C. The needle may be treated at a temperature between 20° C. and 55° C.

In examples, the needle may be treated, for example with the acidic solution, for less than 100 seconds. The needle may be treated for at least 15 seconds. The needle may be treated for between 15 and 90 seconds.

The needle may comprise a proximal portion, for example including a first port of the lumen, and a distal portion, for example including a second port of the lumen. The lumen may comprise a surface which extends between the first port and the second port.

In examples, the lumen surface is treated with the acidic solution at a temperature less than 60° C. for at least 15 seconds.

In examples, following treatment with the acidic solution, for example at a temperature less than 60° C. and/or for at least 15 seconds, the lumen surface has a surface roughness (Rₐ) of less than 5 micrometre. The surface roughness (Rₐ) may be less than 2 micrometres. The surface roughness (Rₐ) may be less than 1 micrometre.

In examples, the needle may be rinsed in a first aqueous solution, for example water.

In examples, the needle may be rinsed in a second aqueous solution, for example a second aqueous solution including an inorganic salt. The inorganic salt may have a pH greater than 7.0. The inorganic salt may include sodium. The inorganic salt may be, for example, sodium carbonate.

In examples, the needle may be boiled in a third aqueous solution, for example water. The third aqueous solution may be demineralized water, also known as demi water, or distilled water. The needle may be boiled in the third aqueous solution at approximately 100° C. The needle may be boiled in the third aqueous solution at a temperature less than 130° C. The needle may be boiled in the third aqueous solution for approximately 60 minutes. The needle may be boiled in the third aqueous solution for between 30 minutes and 90 minutes, for example between 45 minutes and 75 minutes.

In examples, the needle may be dried with filtered air. The needle may be dried with filtered air for less than 60 seconds. The needle may be dried with filtered air for less than 30 seconds, for example less than 20 seconds. The needle may be dried with filtered air for approximately 5 to 10 seconds. The needle may be dried with filtered air at room temperature, for example approximately 20° C. to 22° C. The air may be filtered with a high-efficiency particulate air (HEPA) filter, for example a filter that removes at least 99.9 percent of particles whose diameter is approximately 0.3 micrometres.

In examples, the needle may comprise an outer surface, for example an outer surface. The outer surface may extend between the distal end and the proximal end of the needle. The outer surface may extend between the first port of the lumen and the second port of the lumen of the needle. The outer surface may include less than 10 atomic percent nickel. The outer surface may include less than 5 atomic percent nickel, for example less than 2 atomic percent nickel, for example less than 1 atomic percent nickel.

In examples, the lumen surface may comprise approximately 50 atomic percent nickel. In examples, the lumen surface may comprise at least 48 atomic percent nickel, for example at least 48.5 atomic percent nickel. The lumen surface may comprise less than 52 atomic percent nickel, for example less than 51.2 atomic percent nickel.

In examples, the nickel-titanium alloy may be isobarically annealed in a noble gas at a temperature of at least 300° C. The nickel-titanium alloy may be isobarically annealed in a noble gas at a temperature less than 450° C. The nickel titanium alloy may be isobarically annealed in a noble gas at a temperature between 350° C. and 400° C. The noble gas may be argon.

According to one aspect, there is provided a needle having a lumen for distributing fluid into, or removing fluid from, a soft tissue, for example a subcutaneous tissue, of a patient.

The needle may comprise a proximal portion, a distal portion and an outer surface.

The outer surface may extend between the proximal portion and the distal portion.

The proximal portion may include a port, for example a first port, of the lumen.

The distal portion may include a port, for example a second port, of the lumen.

The outer surface may extend between the first port and the second port.

The needle may comprise a shape-memory material, for example a shape-memory alloy. The needle may comprise, for example, a nickel-titanium alloy.

The outer surface of the needle may comprise less than 10 atomic percent nickel. In examples, the needle may comprise less than 5 atomic percent nickel, for example less than 2 atomic percent nickel. The outer surface of the needle may comprise less than 1 atomic percent nickel.

The low nickel content of the outer surface of the needle advantageously improves the biocompatibility of the needle. In other words, the interaction between the needle and the patient does not result in an inappropriate response, for example an inappropriate response of the patient's immune system.

According to another aspect, there is provided a needle having a lumen for distributing fluid into, or removing fluid from, a soft tissue, for example a subcutaneous tissue, of a patient, the needle comprises: a proximal portion, including a first port of the lumen, a distal portion, including a second port of the lumen, and an outer surface which extends between the first port and the second port; wherein the needle comprises a nickel-titanium alloy, and the outer surface of the needle comprises less than 10 atomic percent nickel.

In examples, the outer surface of the needle may comprise less than 5 atomic percent nickel, for example less than 2 atomic percent nickel. The outer surface of the needle may comprise less than 1 atomic percent nickel.

The low nickel content of the outer surface of the needle advantageously improves the biocompatibility of the needle. In other words, the interaction between the needle and the patient does not result in an inappropriate response, for example an inappropriate response of the patient's immune system.

In examples, the lumen may comprise a surface which extends between the first port and the second port, and the lumen surface may have a surface roughness (Rₐ) of less than 5 micrometres.

The surface roughness (Rₐ) of the lumen surface may be, for example, less than 2 micrometres, for example less than 1 micrometre.

The low surface roughness results in a smooth surface and a larger lumen diameter (for a given wall thickness. Advantageously, this enables better flow of fluids, especially high viscosity fluids, through the needle.

In examples, a wall of the needle may be defined between the outer surface and the lumen surface. The wall may have a thickness which corresponds to the distance between the outer surface and the lumen surface. The wall thickness may be at least 20 micrometres, for example at least 30 micrometres. The wall thickness may be less than 600 micrometres, for example less than 400 micrometres, for example less than 200 micrometres. In some examples, the wall thickness may be approximately 50 micrometres.

In examples, the lumen surface may comprise at least 48 atomic percent nickel, for example at least 48.5 atomic percent nickel. The lumen surface may comprise less than 52 atomic percent nickel, for example less than 51.2 atomic percent nickel. In some examples, the lumen surface may comprise approximately 50 atomic percent nickel.

The nickel-titanium alloy may be superelastic or pseudoelastic.

In examples, the nickel-titanium alloy may be at least 95 percent austenitic at a temperature of at least minus 20° Celsius. The nickel-titanium alloy may be at least 95 percent austenitic at a temperature of at least minus 10° Celsius, for example 0° Celsius, for example 10° Celsius.

In examples, the nickel-titanium alloy may be at least 95 percent austenitic at a temperature of 100° Celsius or less. The nickel-titanium alloy may be at least 95 percent austenitic at a temperature of 75° Celsius or less, for example 50° Celsius or less, for example 40° Celsius or less.

In some examples, the nickel-titanium alloy may be at least 95 percent austenitic at a temperature between approximately 15° C. and approximately 40° C.

In examples, the nickel-titanium alloy is at least 95 percent austenitic following removal of an applied strain of up to 10 percent. The nickel-titanium alloy may be at least 95 percent austenitic following removal of an applied strain of up to 8 percent. The nickel-titanium alloy may be at least 95 percent austenitic following removal of an applied strain of at least 5 percent, for example at least 6 percent.

In examples, the outer surface may comprise an oxide layer. The oxide layer of the outer surface may comprise less than 10 atomic percent nickel.

In examples, the oxide layer of the outer surface may comprise less than 5 atomic percent nickel, for example less than 2 atomic percent nickel. The oxide layer of the outer surface of the needle may comprise less than 1 atomic percent nickel.

In examples, the oxide layer of the outer surface may comprise approximately 60 atomic percent titanium.

In examples, the needle may comprise an outer diameter. The outer diameter may be less than 0.7 millimetres. The outer diameter may be at least 0.4 millimetres.

In examples, the needle may comprise an inner diameter. The inner diameter may be a diameter of the lumen of the needle. The inner diameter, for example the lumen diameter, may be at least 0.2 millimetres. The inner diameter, for example the lumen diameter, may be less than 0.6 millimetres. In an example, the inner diameter, for example the lumen diameter, may be approximately 0.4 millimetres.

According to a further aspect of the invention there is a method of treating a surface of a nickel-titanium needle.

In examples, the method comprises flushing the needle in an acidic solution and/or rinsing the needle and/or boiling the needle. The acidic solution may include hydrofluoric acid.

The method advantageously improves the biocompatibility of the needle, thereby reducing the risk of an inappropriate response, for example an inappropriate response of the patient's immune system to the needle.

According to another aspect of the invention there is provided a method of treating a surface of a nickel-titanium needle, the method comprising: flushing the needle in an acidic solution; rinsing the needle; and boiling the needle.

According to an aspect of the invention there is provided a method of treating a surface of a nickel-titanium needle, the method comprising: flushing the needle in an acidic solution at a temperature less than 60° C.; rinsing the needle; and boiling the needle.

In examples, the needle comprises a lumen, for example a lumen for distributing fluid into, or removing fluid from, a soft tissue, for example a subcutaneous tissue, of a patient. The surface of the needle that is treated may be a lumen surface.

In examples, the surface may be treated, for example with the acidic solution, for less than 100 seconds. The surface may be treated for at least 15 seconds. The surface may be treated for between 15 and 90 seconds. The surface may be, for example, flushed in the acidic solution for at least 10 seconds.

It has been found that, using the relatively quick method, the biocompatibility of the needle is improved. In other words, the interaction between the needle and the patient does not result in an inappropriate response, for example an inappropriate response of the patient's immune system.

In examples, the surface may be treated, for example with the acidic solution, at a temperature of at least 15° C. The surface may be treated at a temperature less than 55° C. The surface may be treated at a temperature between 20° C. and 55° C. The acidic solution may be, for example, at a temperature less than 60° C.

Advantageously, it has been found that the biocompatibility of the needle is improved without the need to process the needle at high temperatures. In this way, the risk of an inappropriate response, for example an inappropriate response of the patient's immune system to the needle is reduced.

In examples, the acidic solution includes hydrofluoric acid. The acidic solution may include at least 5 volume percent hydrofluoric acid. The acidic solution may include less than 50 volume percent hydrofluoric acid. The acidic solution may include, for example between 10 and 45 volume percent hydrofluoric acid.

In examples, the acidic solution includes nitric acid. The acidic solution may include at least 15 volume percent nitric acid. The acidic solution may include less than 70 volume percent nitric acid. The acidic solution may include, for example between 20 and 65 volume percent nitric acid.

Advantageously, it has been found that the method enables the roughness (Rₐ) of the surface of the needle to be reduced to less than 5 micrometres, for example less than 2 micrometres or less than 1 micrometre. This has been found to improve the flow of fluids, particularly viscous fluids, through the needle.

In examples, the needle is rinsed in at least one aqueous solution. The at least one aqueous solution may be water, for example demineralized water, demi water or distilled water.

In examples, the needle is rinsed in a first aqueous solution and a second aqueous solution. The second aqueous solution may include an inorganic salt. The inorganic salt may have a pH greater than 7.0. The inorganic salt may include sodium. The inorganic salt may be, for example, sodium carbonate.

In examples, the needle is boiled in a third aqueous solution. The third aqueous solution may be water. The third aqueous solution may be demineralized water, also known as demi water, or distilled water.

The needle may be boiled in the third aqueous solution at approximately 100° C. The needle may be boiled in the third aqueous solution at a temperature less than 130° C.

The needle may be boiled in the third aqueous solution for approximately 60 minutes. The needle may be boiled in the third aqueous solution for between 30 minutes and 90 minutes, for example between 45 minutes and 75 minutes.

In examples, the needle is dried with filtered air. The needle may be dried with filtered air for less than 60 seconds. The needle may be dried with filtered air for less than 30 seconds, for example less than 20 seconds. The needle may be dried with filtered air for approximately 5 to 10 seconds. The needle may be dried with filtered air at room temperature, for example approximately 20° C. to 22° C. The air may be filtered with a high-efficiency particulate air (HEPA) filter, for example a filter that removes at least 99.9 percent of particles whose diameter is approximately 0.3 micrometres.

In examples, the nickel-titanium needle may be superelastic or pseudoelastic.

The nickel-titanium needle may be isobarically annealed in a noble gas at a temperature of at least 300° C. The nickel-titanium needle may be isobarically annealed in a noble gas at a temperature less than 450° C. The nickel titanium needle may be isobarically annealed in a noble gas at a temperature between 350° C. and 400° C. The noble gas may be argon. In this way, the temperature range over which the superelastic or pseudoelastic properties of the nickel-titanium needle are exhibited may be increased.

For the avoidance of doubt, any of the features described herein apply equally to any aspect of the invention. For example, the method may comprise any one or more features of the needle relevant to the method and/or the needle may comprise any one or more features relevant to one or more features or steps of the method.

### BRIEF DESCRIPTION OF THE SEVERAL VIEWS OF THE DRAWINGS

Examples of the invention are now described, by way of example only, hereinafter with reference to the accompanying drawings, in which:

FIG. 1 shows a side view of an example needle;

FIG. 2 shows a cross-sectional view of the example needle of FIG. 1;

FIG. 3 shows an end view of the example needle of FIG. 1;

FIG. 4 shows a perspective view of the example needle of FIG. 1 mounted to a support member;

FIG. 5 shows a perspective view of an example needle insertion assembly including the support member of FIG. 4;

FIG. 6 shows a graph illustrating certain properties of a nitinol material;

FIG. 7 shows a stress-strain diagram comparing certain properties of a needle formed from nitinol material with those of a needle formed of stainless steel;

FIG. 8 shows a graph illustrating certain properties of needles formed from a nitinol material;

FIG. 9 shows a flow chart of the steps in a method of treating a surface of a nickel-titanium needle;

FIG. 10 shows the relative composition of a nickel-titanium needle following treatment using the method of FIG. 9;

FIG. 11A shows a scanning electron microscope image at 150× magnification of a nickel-titanium needle following treatment using the method of FIG. 9;

FIG. 11B shows a scanning electron microscope image at 2000× magnification of a nickel-titanium needle following treatment using the method of FIG. 9; and

FIG. 11 C shows a scanning electron microscope image at 5000× magnification of a nickel-titanium needle following treatment using the method of FIG. 9.

### DETAILED DESCRIPTION

Certain terminology is used in the following description for convenience only and is not limiting. The words ‘right’, ‘left’, ‘lower’, ‘upper’, ‘front’, ‘rear’, ‘upward’, ‘down’ and ‘downward’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, ‘attached’, ‘coupled’, ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first”, “second”, “third” etc. merely indicate that different instances of like objects are being referred to and are not intended to imply that the objects so described must be in a given sequence, either temporally, spatially, in ranking or in any other manner.

As used herein, elastic deformation refers to the recoverable deformation of at least a portion of a needle so that the portion returns from a deformed, energised state to an un-deformed state.

As used herein, the needle axis is the central longitudinal axis of the needle.

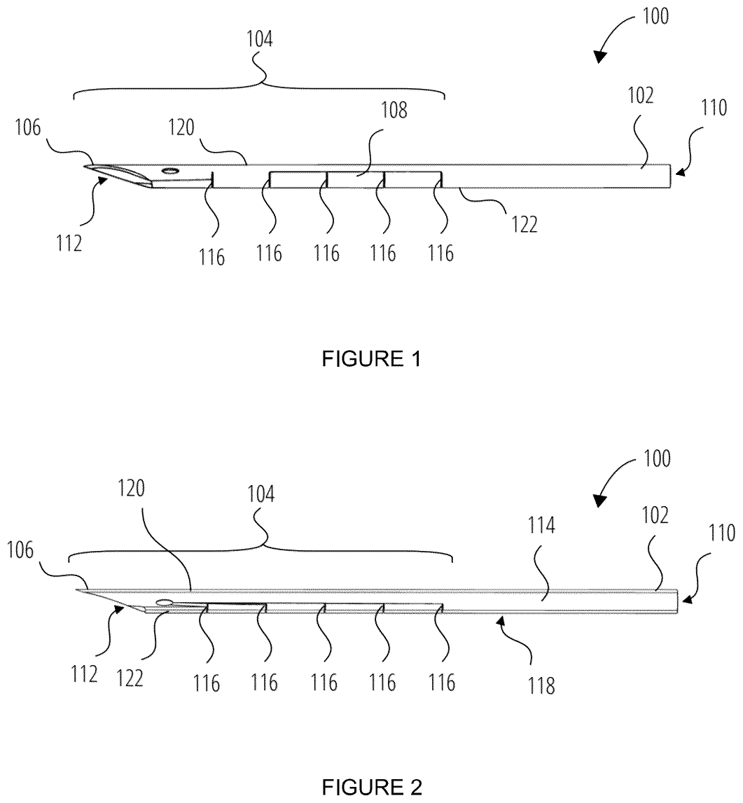

Referring now to FIGS. 1 to 3, there is shown a needle 100, having a lumen 114 for distributing fluid into, or removing fluid from, a soft tissue, for example a subcutaneous tissue of a patient. The needle 100 includes: a proximal portion 102, including a first port 110 of the lumen 114, a distal portion 104, including a second port 112 of the lumen 114, and an outer surface 118, which extends between the first port 110 and the second port 112.

The distal portion 104 includes a sharpened tip 106 provided on a first lengthwise side 120 of the outer surface 118 of the needle 100 and a deformable portion 108 provided on an opposing, second lengthwise side 122 of the outer surface 118 of the needle 100. The tip 106 is configured to incise an entry point in the soft tissue, for example the subcutaneous tissue, of a patient as the tip 106 is urged against the tissue by an urging force provided in a direction along the needle 100. The deformable portion 108 is configured so that, in response to the urging force, the tip 106 is deflectable away from the second lengthwise side 122 of the needle as the tip is inserted into the soft tissue beyond the entry point.

The lumen 114 of the needle 100, particularly the lumen 114 within the distal portion 104, includes a circular cross-section extending within the needle 100. That is, along at least the distal portion 104, in an un-deformed state, the lumen 114 forms a cylindrical tube. The first lengthwise side 120 of the needle and the second lengthwise side 122 of the needle are provided on opposing sides of the cross-section. The lumen 114 has a surface 117 which extends between the first port 110 and the second port 112. The lumen surface 117 forms an inner surface of the needle 100.

In the example shown, the first lengthwise side 120 extends along the needle 100 from the tip 106 to the proximal portion 102. The second lengthwise side 122 extends along the needle 100 to the proximal portion 102 from a region diametrically opposed to the tip 106 of the distal portion 104. Stated differently, with the needle 100 in a straight condition, the first and second lengthwise sides extend along the needle 100 on opposing axial lengths of the outer surface 118 of the needle 100.

The deformable portion 108 of the needle 100 includes a plurality of apertures 116 extending through the needle 100. Each aperture 116 extends from the outer surface 118 of the distal portion 104, through the lumen surface 117 to the lumen 114. A wall 119 of the needle 100 is defined between the outer surface 118 and the lumen surface 117. The lumen 114 is circumscribed by the needle wall 119. Each aperture 116 extends through the needle wall 119.

Each aperture 116 is an elongate slot oriented to extend partially around a circumference of the distal portion 104. In the example, each elongate slot is substantially of equal length and oriented to extend circumferentially, that is perpendicular to the longitudinal axis of the needle 100 in the region of the respective aperture 116. In the example, each elongate slot extends substantially 180° around the circumference of the distal portion.

As will be apparent, other suitable shape, length or orientation of apertures may be provided in the deformable portion 108. In certain examples, the plurality of apertures may each be of varying length. Certain apertures of the plurality of apertures may extend further around the circumference than other apertures. In certain examples, each aperture may be any suitable shape, such as a lozenge shape, ellipse shape. A primary lengthwise axis of each aperture may be oriented at an angle to the longitudinal axis of the needle in the deformable portion 108, for example, oriented at an angle in a range of from 30° to 180°, preferably in a range of from 45° to 150°, more preferably in a range of from 60° to 120°. Certain configurations may include a plurality of helical elongate slots. Certain apertures may be oriented at an acute angle to the longitudinal axis of the needle 100 in the region of the respective aperture 116.

Each aperture 116 of the plurality of apertures is spaced apart along the deformable portion 108 in the lengthwise direction of the needle 100. The apertures 116 are laterally arranged with a spacing of 0.8 mm.

The deformable portion 108 extends along the distal portion 104 for a distance of 4 mm. That is, the deformable portion 108 spans 4 mm along the longitudinal axis of the needle 100 with the plurality of apertures arranged within the span of 4 mm. The apertures are spaced evenly so that each aperture 116 is spaced 0.8 mm apart from neighbouring apertures 116.

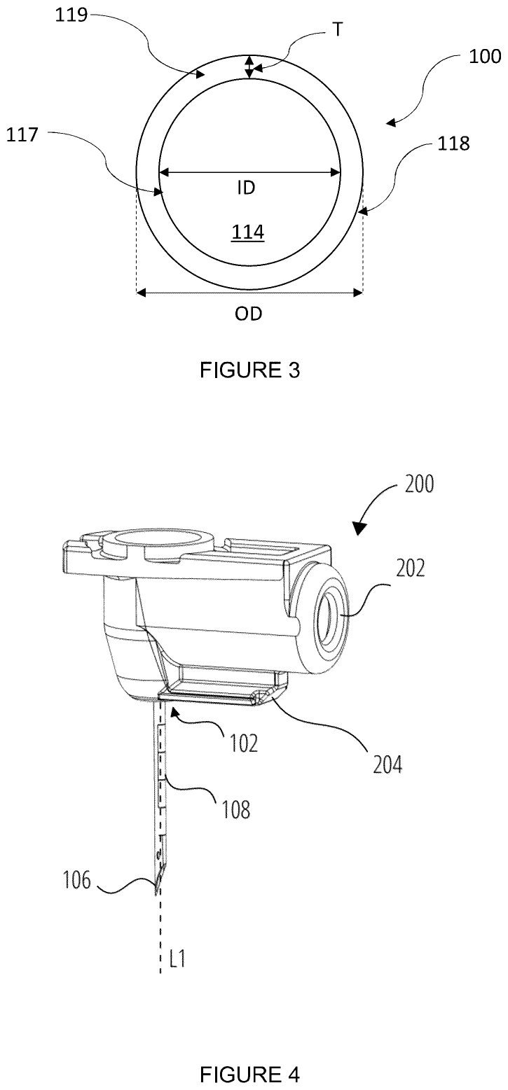

With particular reference to FIG. 3, it can be seen that the needle 100 has an outer diameter (OD) which is defined between the first lengthwise side 120 and the second lengthwise side 122 on the outer surface 118 of the needle 100. The needle 100 also has an inner diameter (ID) which is defined between opposing walls of the surface 117 of the lumen 114 of the needle 100. The wall has a thickness (T) which is corresponds to half the difference between the outer diameter (OD) and the inner diameter (ID) of the needle 100. This can be summarised by the equation T=(OD-ID)/2.

Referring now to FIG. 4, there is shown a support member 200 of a needle insertion assembly including the needle 100 as described with reference to FIGS. 1 to 3. The support member 200 includes a connecting port 202 for delivery or removal of fluid to the needle 100.

The needle 100 is arranged so that its proximal portion is mounted to the support member 200 with the first port is fluidly connected to the connecting port. The needle 100 is mounted to the support member 200 ready for use, so that the needle 100 extends away from a lower surface of the support member 200 along a longitudinal axis L1.

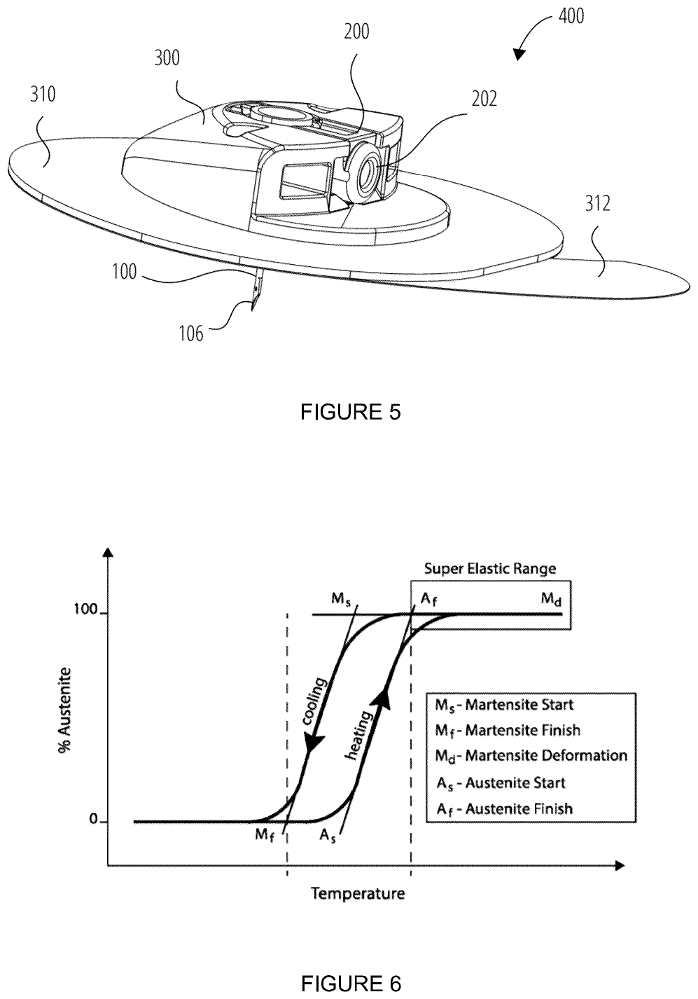

The support member 200 includes a latch element 204. The latch element 204 is configured to lockingly engage with a complementary engaging element (not shown) provided on a base member 300 of a needle insertion assembly 400, an example of which is shown in FIG. 5.

To use the needle 100, the support member 200 is assembled with a receiving portion (not shown) of the base member 300. The support member 200 is loaded into the base member 300 so that the distal portion 104 is inserted, first, through the receiving portion, and then through an opening (not shown). In this way, the receiving portion receivingly engages the support member 200 so that the distal portion 104 of the needle 100 projects outward through the opening.

The support member 200 is mounted to the base member 300 ready for use so that the longitudinal axis L1 of the needle 100 extends away from an engaging surface (not shown) of the base member 300. In particular, in the example, the longitudinal axis L1 is oriented to be perpendicular to the engaging surface when the support member 200 is lockingly engaged with the base member 300 of the needle insertion assembly 400 (for example as shown in FIG. 5).

The needle insertion assembly 400 is shown in an assembled configuration, with the support member 200 lockingly engaged with the base member 300, but without affixing the needle insertion assembly 400 to soft tissue. The needle insertion assembly 400 includes an adhesive patch 310. The adhesive patch 310 is suitably tacky to affix a lower surface of the base member 300, typically the engaging surface, to the soft tissue of the patient. With the needle insertion assembly 400 affixed to the patient, the support member 200, including the needle 100, may be mounted to the needle insertion assembly 400 as described herein, so that the needle 100 is inserted into the soft tissue of the patient. As will be appreciated, the needle insertion assembly 400 may be affixed or otherwise secured to the patient without an adhesive patch 310, instead using other suitable means.

In the example, as shown particularly in FIG. 5, a removable strip 312 is located over the adhesive patch 310. The removable strip 312 is removed from the adhesive patch 310 when the base member 300 is to be affixed to the soft tissue.

The adhesive patch 310 includes through-hole. The adhesive patch 310 is suitably positioned to align the through-hole with the opening on the engaging surface of the base member 300 when the adhesive patch 310 is affixed to the lower surface.

The needle insertion assembly 400 may optionally include a protective cover (not shown) mountable to, or around, a perimeter surface of the base member 300. The protective cover may be mounted to the base member 300 after the base member 300 is affixed to a patient's soft tissue, enclosing perimeter surface of the base member 300 against the soft tissue. The protective cover thereby reduces ingress of dirt or contamination between the soft tissue and the needle insertion assembly 400 during use.

The needle 100 and needle insertion assembly 400 described herein provide a convenient method of inserting a needle having a lumen for distributing fluid into, or removing fluid from, a soft tissue, for example a subcutaneous tissue, of a patient. According to a first step, the method includes providing a needle 100 having a lumen 114, the needle 100 including a proximal portion 102 and a distal portion 104, wherein the distal portion 104 includes: a sharpened tip 106 provided on a first lengthwise side 120 of the needle 100, and a deformable portion 108 provided on an opposing, second lengthwise side 122 of the needle 100.

According to another step, the method includes urging the tip 106 against the soft tissue with an urging force provided in a direction along the needle 100 to incise an entry point in the soft tissue of a patient.

According to a further step, the method includes deflecting the tip 106 away from the second lengthwise side 122 of the needle 100 as the tip 106 is inserted into the soft tissue beyond the entry point. In the example shown, the method includes deflecting the tip 106 away from the second lengthwise side 122 of the needle 100 at an angle of 30°.

In certain embodiments, the needle 100 may be superelastic, i.e. the needle may be formed from a superelastic material, for example a superelastic alloy. Superelasticity, also referred to as pseudo-elasticity, is an elastic response exhibited by certain materials to an applied stress. Superelasticity occurs when an applied stress induces an austenite to martensite phase transformation in the material and a corresponding strain, which is recoverable by removing the applied stress. Certain superelastic materials exhibit recoverable strains of up to 11%, which is significantly greater than more conventional materials. For example, 316 stainless steel (16% chromium, 10% nickel and 2% molybdenum), which is commonly used in medical applications, exhibits recoverable strains of approximately 0.5%. A superelastic alloy used in medical applications is nickel titanium alloy, commonly referred to as nitinol. The needle 100 may comprise a nickel-titanium alloy. The needle 100 may be formed of a nickel-titanium alloy, for example nitinol. As superelasticity is a stress-induced phase transformation from austenite to martensite, for the needle 100 to exhibit optimum superelasticity it may be formed of so-called austenitic (or superelastic) nitinol, in which nitinol will be substantially fully austenitic, i.e. the primary crystalline structure of the alloy is austenite. Nitinol will remain substantially fully austenitic above its martensite start temperature Mₛ. This is important to note because an austenite to martensite phase transformation can be induced by cooling as well by applied stress.

Referring now to FIG. 6, there is shown a temperature-induced phase transformation of a nitinol alloy between the austenitic and martensitic phases, in which austenite is stable at relatively higher temperatures and martensite is stable at relatively lower temperatures. Heating nitinol beyond its austenite start temperature Aₛ causes it to transform to the austenitic phase. Nitinol will be substantially fully austenitic once heated above its austenite finish temperature A_{f}. As above, it is in this substantially fully austenitic phase that nitinol will exhibit optimum superelasticity, allowing the needle when formed of nitinol to elastically deform, i.e. flex/bend, through a relatively broad range of stresses without causing permanent deformation. The optimal superelastic range (also referred to as a superelastic window) between the austenite finish temperature A_{f} and the martensite deformation temperature M_{d}, is highlighted in FIG. 6. From the substantially fully austenitic phase, cooling nitinol beyond its martensite start temperature Mₛ causes nitinol to transition to the martensitic phase. Below its martensite finish temperature M_{f} nitinol will be substantially fully martensitic.

The graph in FIG. 6 also shows that nitinol exhibits thermal hysteresis, i.e. the temperature at which martensite transforms to austenite is not that at which austenite transforms to martensite. The hysteresis may be approximately 20-30° C. (i.e. A_{f}-M_{f}) for fully annealed nitinol alloys, such as those used in medical device applications. It is known that a greater thermal hysteresis will yield a greater mechanical hysteresis. The significance of its thermal hysteresis is that nitinol remains in its austenitic phase when cooled beyond its austenite finish temperature A_{f}. This means a needle, for example the needle 100, when formed of austenitic nitinol will remain superelastic above its martensite start temperature Mₛ, which may therefore be the critical transformation temperature when selecting an alloy forming the needle.

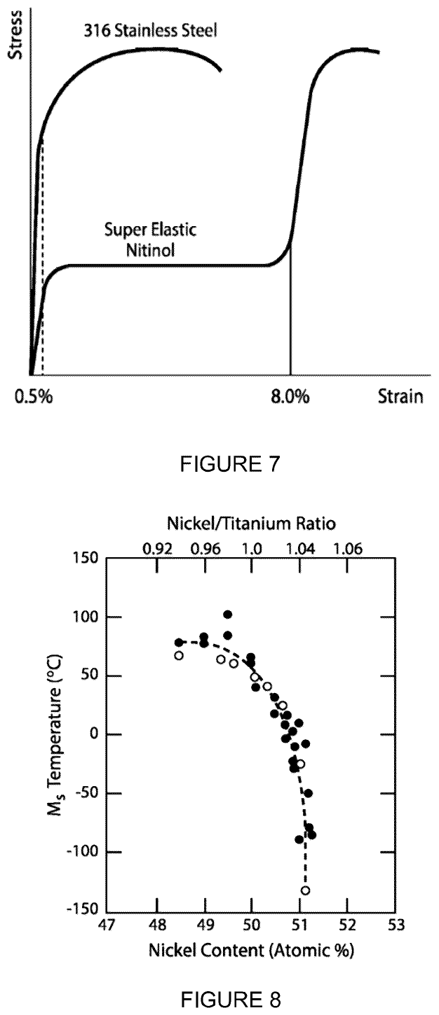

Referring now to FIG. 7, there is shown a graph comparing the behavior of a needle formed of 316 stainless steel with a needle, for example the needle 100, comprising a nickel-titanium alloy. As is evident from FIG. 7, the nitinol needle has a large range of elastic deformation in which increased strain does not significantly increase the stresses within the needle. In the illustrated form, stresses begin to rise at about 8% strain. As such, it may be advisable to maintain the strain associated with the needle below about 6% in order to ensure that the needle remains within its elastic deformation range. That is, it may be advisable to maintain the strain associated with the needle below about 6% in order to ensure that the needle remains below its elastic deformation limit.

Referring now to FIG. 8, there is shown a correlation between the martensite start temperature Mₛ and the composition of nitinol. Since the martensite start temperature Mₛ is correlated with the nickel/titanium ratio it can be predetermined, for example a nitinol alloy can be selected having a martensite start temperature Mₛ from approximately −130° C. to approximately +110° C.

Certain embodiments of the invention may have particular application for use in devices for continuous subcutaneous insulin infusion (CSII) therapy, in which the devices may be worn by a patient such that at least the tip 106 and the distal portion 104 of the needle 100 is placed in the patient's soft tissue. Due to the superelasticity of nitinol, the needle 100 may reduce tissue damage by exhibiting a relatively high degree of flexibility. However, if the martensite percentage of the material forming the tip begins to rise, exposure to stress may cause it undergo plastic deformation, resulting in an irregular, uncontrolled shape change. This may lead to tissue damage. While the patient's body heat may aid in maintaining nitinol in its austenitic phase, certain embodiments of the needle 100 may be placed in tissue to a relatively low depth, such as approximately 3 to 4 mm. As such, cold temperatures experienced by the patient may cause a temperature-induced phase transformation of the nitinol alloy forming the needle to its martensitic phase, i.e. if the martensite start temperature Mₛ is too high. Thus, in certain embodiments, the needle may be formed of a nitinol alloy selected to remain substantially fully austenitic (for example, at least 95% austenitic) in temperatures likely to be experienced by the patient to reduce the risk of the needle becoming martensitic.

As indicated in FIG. 8, superelasticity is exhibited up to the martensite deformation temperature M_{d}, which corresponds to the greatest temperature at which it is possible to stress-induce the formation of martensite. Above the martensite deformation temperature M_{d} the response to stress is non-elastic deformation of the austenitic microstructure, since martensite can no longer be formed, and thus permanent deformation. In other words, above the martensite deformation temperature M_{d} the nitinol will deform plastically and irreversibly.

The effective superelastic window of the nitinol may be increased to span more than 200° C., with a significant reduction in temperature-stress sensitivity, by subjecting the material to a controlled process. The nitinol may, for example, be subjected to an annealing process or treatment, e.g. an isobaric annealing process or treatment in Argon at approximately 350° C. to 400° C. In this way, the martensite deformation temperature M_{d} of the material is increased and the effective superelastic window or range may be increased or widened.

In view of the above, the needle 100 may be formed of a nitinol alloy having been subjected to an isobaric annealing treatment in order to increase the effective superelastic window in order to ensure that elastic deformation through a relatively broad range of stresses is possible. In this way the needle can be manufactured from nitinol that elastically bends without permanent deformation of the needle. The needle may also be formed of a nitinol alloy having an austenite finish temperature A_{f} in the range of approximately −15° C. to approximately 20° C. in order to ensure that the needle has good superelastic properties at body temperature (approximately 37° C.).

More generally, the needle 100 may comprise a nickel-titanium alloy selected such that the needle 100, or part therefore, exhibits selected properties and/or characteristics with reference to a particular temperature to be experienced by the needle 100. In certain embodiments, the particular temperature may be body temperature (approximately 37° C.) or room temperature (for example, approximately 20° C. to 22° C.).

FIG. 8 also shows that nitinol is typically composed of approximately 50 to 51 % nickel by atomic percent, although compositions outside this range are known, and various compositions may be suitable for forming the needle. In certain embodiments, an atomic ratio of nickel to titanium is between 1.01 and 1.05. In certain embodiments, an atomic ratio of nickel to titanium is between 1.02 and 1.04.

The needle formed of austenitic (or superelastic) nitinol may exhibit greater flexibility than a similarly-configured needle formed of a more conventional material, such as stainless steel. The skilled reader will understand that the range of superelasticity for a particular composition of nitinol depends largely upon its nickel/titanium ratio. In certain embodiments, the needle may be formed of nitinol having a nickel/titanium ratio selected to provide the needle with particular properties and/or characteristics. The skilled reader will also understand that the range of superelasticity of the nitinol also depends on how the material has been processed. In certain embodiments, the needle may be formed of nitinol that has been processed or treated in a controlled way in order to provide the needle with particular properties and/or characteristics.

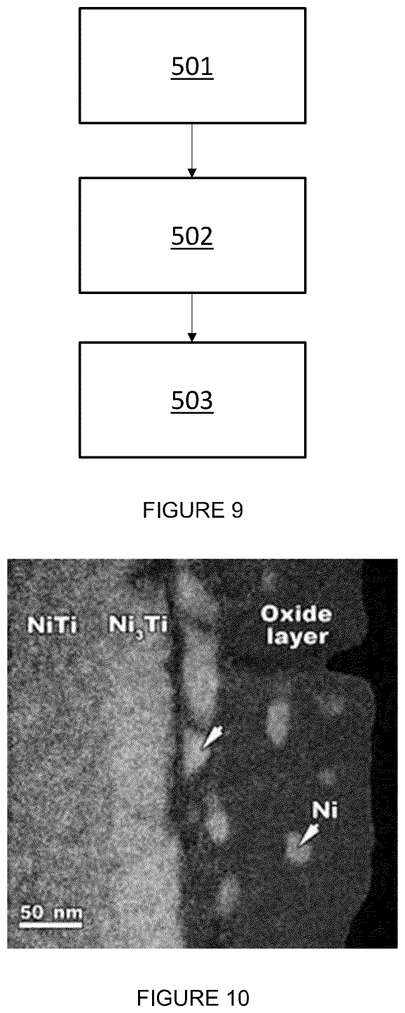

A method of treating a surface of the nickel-titanium needle 100 will now be described with reference to FIG. 9.

The method includes flushing the needle 100 with an acidic solution in step 501, rinsing the needle 100 in step 502 and boiling the needle 100 in step 503.

In step 501, an acidic solution including hydrofluoric acid and nitric acid is prepared. The acidic solution may be, for example, prepared by mixing 2 parts water with 1 part Polinox B. The acidic solution may thus include approximately 45 volume percent hydrofluoric acid and approximately 20 volume percent nitric acid.

The needle 100 is connected to a syringe via a tube or hose. The acidic solution (approximately 1.5 millilitres) is sucked up through the needle 100. After a venting period (approximately 30 seconds), approximately 0.5 milliltres of the acidic solution is expelled. After a further venting period (approximately 10 seconds), a further approximately 0.5 millilitres of the acidic solution is expelled. After another venting period (approximately 50 seconds), the remaining acidic solution (approximately 0.5 millilitres) is expelled from the needle.

In this way the lumen surface 117 is treated with the acidic solution for approximately 90 seconds.

It has been found that the surface treatment is particularly effective if carried out between 20 and 30 degrees Celsius. In other words, the temperature of the acidic solution is maintained at between 20 and 30 degrees Celsius.

The needle 100 is then rinsed in step 502. The needle 100 is initially rinsed with a first aqueous solution, for example water. The water is flushed into and out of the needle 100 using a syringe. In examples of the method, the water is flushed into and out of the needle 100 three times.

A second aqueous solution including 1 weight percent sodium carbonate in water is then prepared. The second aqueous solution (approximately 1.5 millilitres) is sucked up into the needle 100. After a venting period (approximately 10 seconds), the second aqueous solution is expelled from the needle 100.

One or more further rinse steps may then be carried out using a third aqueous solution, for example demineralized water and/or boiled demineralized water.

In a first further rinse step, the demineralized water is flushed into and out of the needle 100 using a syringe. In examples of the method, the demineralized water is flushed into and out of the needle 100 three times.

In a second further rinse step, the demineralized water is flushed into and out of the needle 100 using a syringe. In examples of the method, the demineralized water is flushed into and out of the needle 100 once.

In a third further rinse step, the boiled demineralized water is flushed into and out of the needle 100 using a syringe. In examples of the method, the boiled demineralized water is flushed into and out of the needle 100 once.

In a fourth further rinse step, the boiled demineralized water is flushed into the needle 100 using a syringe. The syringe is then removed from the needle 100, and the needle 100 is placed in demineralized water.

In step 503, the needle 100 is boiled in demineralized water at approximately 100 degrees Celsius for approximately 60 minutes.

The needle 100 is then dried with filtered air (which, for example, has been filtered with a high-efficiency particulate air (HEPA) filter at a temperature of approximately 20 to 22 degrees Celsius for approximately 15 seconds and packaged in a heat-sealed bag for storage.

Using the method described above, it has been found that it is possible to chemically remove the oxide layer on the outer surface 118 of the needle 100 and then form a titanium oxide layer (i.e. an oxide layer with no, or at least a very small quantity, of nickel) on the outer surface 118 of the needle 100, as illustrated in FIG. 10. The outer surface 118 of the needle 100 may, for example, comprise less than 1 atomic percent nickel.

The layer of the outer surface 118 of the needle 100 which has no, or at least a very small quantity of, nickel may have a depth of less than 150 nm, for example less than 125 nm.

The layer of the outer surface 118 of the needle 100 which has no, or at least a very small quantity of, nickel may have a depth of at least 25 nm, for example, at least 50 nm.

The layer of the outer surface 118 of the needle 100 which has no, or at least a very small quantity of, nickel may have a depth of between 25 nm and 150 nm, for example, between 25 nm and 125 nm. The layer of the outer surface 118 of the needle 100 which has no, or at least a very small quantity of, nickel may have a depth of between 50 nm and 150 nm, for example, between 50 nm and 125 nm.

The nickel content of the outer surface 118 of the needle 100 may be determined using any suitable method, for example the methods described in British Standard ISO 22309:2011, Microbeam analysis. Quantitative analysis using energy-dispersive spectrometry (EDS) for elements with an atomic number of 11 (Na) or above.

Advantageously, this results in a nickel-titanium needle 100 that has beneficial superelastic properties and which has improved biocompatibility.

The surface roughness (Rₐ) of the inner surface 117 and the outer surface 118 of the needle may be determined using any suitable method, for example the methods described in British Standard ASME B46.1-2019, Surface Texture (Surface Roughness, Waviness and Lay).

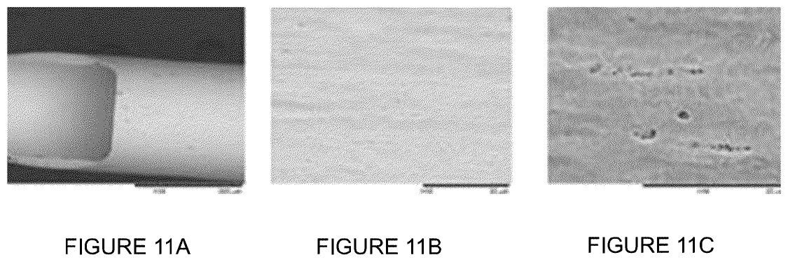

It has also been found that the method results in the lumen surface 117 of the needle 100 having a surface roughness (Rₐ) of approximately 1 micrometre, as shown in FIGS. 11A to 11C.

The low surface roughness results in a smooth surface and a larger lumen diameter (for a given wall thickness. Advantageously, this enables better flow of fluids, especially high viscosity fluids, through the needle 100.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A needle having a lumen for distributing fluid into, or removing fluid from, a soft tissue of a patient, the needle comprising a nickel-titanium alloy; wherein the needle is treated with an acidic solution including hydrofluoric acid at a temperature less than 60° C. for at least 10 seconds.

2. A needle according to claim 1, wherein the acidic solution includes at least 5 volume % hydrofluoric acid.

3. A needle according to claim 1 or claim 2, wherein the acidic solution includes less than 50 volume % hydrofluoric acid.

4. A needle according to claim 1, claim 2 or claim 3, wherein the acidic solution also includes nitric acid.

5. A needle according to claim 4, wherein the acidic solution includes at least 20 volume % nitric acid.

6. A needle according to any of claims 1 to 5, wherein the temperature is at least 15° C.

7. A needle according to any of claims 1 to 6, wherein the needle is treated with the acidic solution for less than 100 seconds.

8. A needle according to any of claims 1 to 7, the needle comprising: a proximal portion, including a first port of the lumen, and a distal portion, including a second port of the lumen, wherein the lumen comprises a surface which extends between the first port and the second port; and the lumen surface is treated with the acidic solution at a temperature less than 60° C. for at least 15 seconds.

9. A needle according to claim 8, wherein, following treatment with the acidic solution at a temperature less than 60° C. for at least 15 seconds, the lumen surface has a surface roughness (Ra) of less than 5 μm.

10. A needle according to any of claims 1 to 9, wherein the needle is rinsed in a first aqueous solution.

11. A needle according to claim 10, wherein the needle is rinsed in a second aqueous solution, the second aqueous solution including an inorganic salt.

12. A needle according to claim 11, wherein the inorganic salt has a pH greater than 7.0.

13. A needle according to any of claims 1 to 12, wherein the needle is boiled in a third aqueous solution at approximately 100° C. for approximately 60 minutes.

14. A needle according to any of claims 1 to 13, wherein the needle is dried with filtered air.

15. A needle according to any of claims 1 to 14, the needle comprising an outer surface extending between the first port of the lumen and the second port of the lumen, wherein the outer surface includes less than 10 atomic % nickel.

16. A needle according to any of claims 1 to 15, wherein the lumen surface comprises approximately 50 atomic % nickel.

17. A needle according to any of claims 1 to 16, wherein the nickel-titanium alloy is isobarically annealed in argon at a temperature of at least 300° C.

18. A method of treating a surface of a nickel-titanium needle, the method comprising: flushing the needle in an acidic solution including hydrofluoric acid; rinsing the needle; and boiling the needle.

19. A method according to claim 18, wherein the surface is a lumen surface.

20. A method according to claim 18 or claim 19, wherein the needle is flushed in the acidic solution for at least 10 seconds.

21. A method according to claim 18, claim 19 or claim 20, wherein the acidic solution is at a temperature less than 60° C.

22. A method according to any of claims 18 to 21, wherein the acidic solution includes hydrofluoric acid.

23. A method according to claim 22, wherein the acidic solution includes nitric acid.

24. A method according to any of claims 18 to 23, wherein the needle is rinsed in at least one aqueous solution.

25. A method according to claim 24, wherein the needle is rinsed in a first aqueous solution and a second aqueous solution.

26. A method according to claim 25, wherein the second aqueous solution includes an inorganic salt.

27. A method according to claim 26, wherein the inorganic salt has a pH greater than 7.0.

28. A method according to claim 26 or claim 27, wherein the needle is boiled in a third aqueous solution.

29. A method according to claim 28, wherein the needle is boiled in the third aqueous solution at approximately 100° C. for approximately 60 minutes.

30. A method according to any of claims 18 to 29, wherein the needle is dried with filtered air.

31. A needle having a lumen for distributing fluid into, or removing fluid from, a soft tissue of a patient, the needle comprising:
a proximal portion, including a first port of the lumen,
a distal portion, including a second port of the lumen, and
an outer surface which extends between the first port and the second port;
wherein the needle comprises a nickel-titanium alloy, and
the outer surface of the needle comprises less than 10 atomic % nickel.

32. A needle according to claim 31, wherein the lumen comprises a surface which extends between the first port and the second port; and
the lumen surface has a surface roughness (Rₐ) of less than 5 μm.

33. A needle according to claim 32, wherein a wall of the needle is defined between the outer surface and the lumen surface; and
the wall has a thickness that is at least 20 μm.

34. A needle according to claim 33, wherein the wall thickness is less than 400 μm.

35. A needle according to claim 32, claim 33 or claim 34, wherein the lumen surface comprises approximately 50 atomic % nickel.

36. A needle according to any one of claims 31 to 35, wherein the nickel-titanium alloy is at least 95% austenitic at a temperature of at least −20° C.

37. A needle according to claim 36, wherein the nickel-titanium alloy is at least 95% austenitic at a temperature of 100° C. or less.

38. A needle according to any one of claims 31 to 37, wherein the nickel-titanium alloy is at least 95% austenitic following removal of an applied strain of up to 10%.

39. A needle according to any one of claims 31 to 38, wherein the outer surface comprises an oxide layer; and
the oxide layer comprises less than 10 atomic % nickel.

40. A needle according to claim 39, wherein the oxide layer comprises approximately 60% titanium.

41. A needle according to any one of claims 31 to 40, the needle comprising an outer diameter;
wherein the outer diameter is less than 0.7 mm.

42. A needle according to claim 41, wherein the outer diameter is at least 0.4 mm.

43. A needle according to any one of claims 31 to 42, the needle comprising an inner diameter;
wherein the inner diameter is at least 0.2 mm.

44. A needle according to claim 43, wherein the inner diameter is less than 0.6 mm.
